(19) United States
(12) Patent Application Publication
(10) Pub. No.: US 2026/0027175 A1
(43) Pub. Date: Jan. 29, 2026

(54) COMPOSITIONS AND METHODS FOR TREATING A COGNITIVE DEFICIT

(71) Applicant: DR DOZO LABORATORIES, Mohali, Punjab (IN)

(72) Inventor: Gurdial Singh ARORA, Mohali, Punjab (IN)

(21) Appl. No.: 18/997,057

(22) PCT Filed: Aug. 10, 2023
(86) PCT No.: PCT/IN2023/050768

§ 371 Date: Jan. 18, 2025

(30) Foreign Application Priority Data
Aug. 10, 2022 (IN) 202211045802

(51) Int. Cl. A61K 36/8998; A61P 25/28

(52) U.S. Cl. CPC A61K 36/8998; A61P 25/28

(57) ABSTRACT

The present disclosure relates to compositions and methods for preventing or treating a cognitive deficit in a subject. The compositions comprise a barley extract as an active ingredient.

### CROSS-REFERENCE TO RELATED APPLICATIONS

This application claims the benefit of priority to Indian Provisional Patent Application No. 20/221,1045802, filed on Aug. 10, 2023, which is hereby incorporated by reference herein in its entirety.

### TECHNICAL FIELD

The present disclosure relates to compositions and methods for treating a cognitive deficit.

### BACKGROUND

Tau proteins are microtubule-associated proteins in neurons and under physiological conditions, tau plays a key role in microtubule stabilization, axonal transportation and neurite outgrowth (Yi and Simpkins, 2008). The disruption of normal phosphorylation events resulting into deposits of abnormally hyperphosphorylated tau protein in intraneuronal neurofibrillary tangles (NFTs) represents an important neuropathological hallmark in Alzheimer's disease (AD) brain (Ballatore et al., 2007).

Previous studies reveal that down-regulation of protein phosphatases or upregulation of protein kinases are the main causes for tau hyperphosphorylation. The hyperphosphorylated tau had a reduced ability to bind to microtubules and to promote microtubule assembly in-vitro (Ballatore et al., 2012). Hyperphosphorylation of tau by the reduced activity of protein phosphatases 1/2A (PP2A) in-vitro suggests a crucial role of PP2A in tangle formation in AD (Ballatore et al., 2007).

Okadaic acid (OKA) is an oxidative inducer and a selective and potent inhibitor of serine/threonine phosphatases 1 and 2A, thus inducing hyperphosphorylation of tau in-vitro and in-vivo (Benitez-King et al., 2003).

It is present in dinoflagellates (Helicondria Okadai), and it has been observed that the population consuming dinoflagellate contaminated sea-food suffers from memory impairment (www.aristatek.com/Newsletter/DEC07/DEC07ts.aspx) (Cohen, 1990; Rajasekar et al., 2013). Literature provides substantial evidence that OKA-induced tau hyperphosphorylation coupled with neuroinflammation and oxidative stress represent an effective experimental paradigm of AD.

Oxidative stress and neuroinflammation play pivotal role in pathogenesis of AD. OKA also increases oxidative stress in the brain, which is characterized by high level of lipid peroxidation, decreases in GSH and diminished activities of antioxidative enzymes (Tunez et al., 2003).

### SUMMARY

Provided herein is a composition comprising barley extract as an active ingredient for (i) preventing or treating a cognitive deficit, (ii) preventing or treating a condition, a disease, or a disorder, wherein inhibition of JNK (Jun N-terminal kinase) provides a benefit, and/or iii) preventing or treating a condition, a disease, or a disorder associated with Tau-protein activity, in a subject.

In another aspect, the present disclosure provides use of a composition comprising barley extract as an active ingredient in the manufacture of a medicament for (i) preventing or treating a cognitive deficit, (ii) preventing or treating a condition, a disease, or a disorder, wherein inhibition of JNK provides a benefit, and/or iii) preventing or treating a condition, a disease, or a disorder associated with Tau-protein activity, in a subject.

In yet another aspect, the present disclosure provides a method for (i) preventing or treating a cognitive deficit, (ii) preventing or treating a condition, a disease, or a disorder, wherein inhibition of JNK provides a benefit, and/or (iii) preventing or treating a condition, a disease, or a disorder associated with Tau-protein activity, in a subject. The method comprises administering to the subject a composition comprising a barley extract as an active ingredient.

### BRIEF DESCRIPTION OF DRAWINGS

FIG. 1 illustrates experimental design for testing effect of composition according to an embodiment of the present disclosure in animals.

FIG. 2 illustrates treatment effect of composition according to the present disclosure on % Initial Transfer Latency (% ITL) of ICV-OKA rats in Morris water maze; values are expressed as mean±SEM; and a represents difference from sham group (p<0.05); and b represents difference from ICV-OKA administered group (p<0.05).

FIG. 3 illustrates treatment effect of composition according to the present disclosure on NF-Kβ activity in ICV-OKA rats; values are expressed as mean±SEM; a represents difference from sham group (p<0.05); b represents difference from ICV-OKA administered group (p<0.05).

FIG. 4 illustrates treatment effect of composition according to the present disclosure on BDNF level in ICV-OKA rats; values are expressed as mean±SEM; a represents difference from sham group (p<0.05); and b represents difference from ICV-OKA administered group (p<0.05).

FIG. 5 illustrates example of MAP2 staining and Phospho-Tau staining of control conditions: No treated, okadoic acid 100 nM (3 H) and okadoic acid 100 nM after Lithium Chloride treatment for 24 H.

FIG. 6 illustrates quantification of Phospho-Tau staining in the neutrons of conditions after 24 H treatment. The statistical analysis is Student t-test *p<0.05; **p<0.01.

FIG. 7 illustrates example of Phospho-Tau staining of SAAC treated cells for 24 H followed by okadoic acid treatment for 3 H.

FIG. 8 illustrates example of MAP2 staining of SAAC treated cells for 24 H followed by okadoic acid treatment for 3 H.

FIG. 9 illustrates quantification of Phospho-Tau staining in the neutrons of SAAC treated cells after a 24 H treatment followed by okadoic acid treatment (10 nM or 100 nM) for 3 H. The statistical analysis is a Student t-test *p<0.05.

FIG. 10 illustrates Phospho-Tau staining of SAAC treated cells for 24 H followed by okadoic acid treatment for 3 H. The yellow arrays identify the P-Tau staining in the neuropil (dense axone and dendrites network between the neutrons).

FIG. 11 illustrates quantification of Phospho-Tau staining in the neutrons of controlled conditions after 4 H treatment. The statistical analysis is a Student t-test: *p<0.05; **p<0.01.

FIG. 12 illustrates quantification of Phospho-Tau staining in the neutrons of SAAC treated cells after a 4 H treatment followed by okadoic acid treatment (10 nM or 100 nM) for 3 H. The statistical analysis is a Student t-test: *p<0.05.

FIG. 13 illustrates (A) image sample of the phosphor c-Jun staining in the untreated and the aniosomycin reference compound conditions; (B) effect of SACC and aniosomycin on the level of c-Jun phosphorylation.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term ‘compound(s)’ comprises the compounds disclosed in the present invention.

As used herein, the term “comprises” or “comprising” is generally used in the sense of include, that is to say permitting the presence of one or more features or components.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as the event or circumstance where the alkyl is not substituted.

As used herein, the term “prevents”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “therapeutically effective amount” refers to that amount of the active ingredient (e.g. barley extract) being administered sufficient to prevent development of or alleviate to some extent one or more of the symptoms of the condition or disorder being treated.

As used herein, the term “treat”, “treating” and “treatment” refer to a method of alleviating or abrogating a disease and/or its attendant symptoms.

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus, it is intended that the present invention includes such modifications and variations and their equivalents. Other objects, features and aspects of the present invention are disclosed in or are obvious from, the following detailed description. It is to be understood by one of ordinary skill in the art that the present discussion is a description of exemplary embodiments only and is not to be construed as limiting the broader aspects of the present invention.

In an embodiment, the present disclosure provides a composition comprising barley extract as an active ingredient for use in (i) preventing or treating a cognitive deficit, (ii) preventing or treating a condition, a disease, or a disorder, wherein inhibition of JNK (Jun N-terminal kinase) provides a benefit, and/or iii) preventing or treating a condition, a disease, or a disorder associated with Tau-protein activity, in a subject. The barley extract may be extracted by any method known in the art. In certain embodiments, the barley extract is obtained by the method described in Indian patent number 236375, the entirety of which is incorporated herein by reference.

In certain embodiments, the present disclosure provides a composition comprising barley extract as an active ingredient for preventing or treating a cognitive deficit in a subject. In some embodiments, the composition comprising barley extract as an active ingredient is for use in preventing or treating a cognitive deficit in a subject. The barley extract may be extracted by any method known in the art. In certain embodiments, the barley extract is obtained by the method described in Indian patent number 236375, the entirety of which is incorporated herein by reference.

In certain embodiments, the present disclosure provides a composition comprising barley extract as an active ingredient for preventing or treating a cognitive deficit in a subject, wherein the barley extract is obtained by the process comprising the steps of mixing barley grain flour with distilled water to get a mixture; stirring the mixture at an ambient temperature; distilling the mixture to obtain a distilled extract; and filtering the distilled extract to obtain the barley extract. In some instances of the extraction process, the mixture is kept at a temperature of about 27+3° C. for about 16+2 hours. In certain embodiments, the mixture is distilled at a temperature of about 110+30° C. to obtain the extract. The distilled extract thus obtained is kept at about 10+3° C. for about 1 h. Next, the extract is brought to room temperature and filtered. The filtration may be done using any suitable method, for example using a filter press or a belt filter or a vacuum rotary filter. In certain embodiments, the extract is filtered over a bed of cotton, filter paper, or filter cloth. In some instances, the extract is filtered over a bed of cotton. In certain embodiments, the obtained filtrate (also referred to interchangeably herein as “barley extract”) is labeled and stored at about 4 to about 8° C. for a longer shelf-life. In certain embodiments, the obtained barley extract may be directly used as an active agent for preventing or treating a cognitive deficit in a subject. In some embodiments, the obtained barley extract may be further concentrated and used, or may be dried to remove water as necessary. The extract can be concentrated by any method known in the art.

In certain embodiments, the cognitive deficit is associated with the activity of Tau-protein, JNK, BDNF and/or NF-κβ. In some embodiments, the cognitive deficit is associated with the activity of Tau-protein.

In certain embodiments, the composition provided herein may be used as a pharmaceutical composition or a food composition. The food composition, in addition to the barley extract, may further comprise one or more additives that are generally used in a food composition.

In certain embodiments, the present disclosure provides a pharmaceutical composition comprising barley extract as an active ingredient for preventing or treating a cognitive deficit in a subject. In some embodiments, the cognitive deficit is associated with the activity of Tau-protein, JNK, BDNF and/or NF-κβ.

In certain embodiments, the present invention provides a pharmaceutical composition comprising barley extract as an active ingredient for preventing or treating a condition, a disease, or a disorder in a subject, wherein inhibition of JNK provides a benefit. In some embodiments, the condition, the disease, or the disorder is a cognitive deficit. In further embodiments, the cognitive deficit is protein phosphatase inhibitor induced cognitive deficit. In some instances, the protein phosphatase inhibitor is okadaic acid (OKA) or a pharmaceutically acceptable salt thereof. In further instances, the cognitive deficit is Alzheimer's disease (AD) or Parkinson's disease. Thus, in certain embodiments, the pharmaceutical composition comprising barley extract as an active ingredient is useful in preventing or treating Alzheimer's disease associated with the activity of JNK, in a subject. The barley extract may be prepared in the same manner as described above or in Example 1 below.

In certain embodiments, the present disclosure provides a pharmaceutical composition comprising barley extract as an active ingredient for preventing or treating a condition, a disease, or a disorder associated with Tau-protein activity in a subject. In some embodiments, the condition, the disease, or the disorder is a cognitive deficit. In further embodiments, the cognitive deficit is protein phosphatase inhibitor induced cognitive deficit. In some instances, the protein phosphatase inhibitor is okadaic acid (OKA) or a pharmaceutically acceptable salt thereof. In further instances, the cognitive deficit is Alzheimer's disease (AD) or Parkison's disease. Thus, in certain embodiments, the pharmaceutical composition comprising barley extract as an active ingredient is useful in preventing or treating Alzheimer's disease associated with the activity of Tau-protein, in a subject. The barley extract may be prepared in the same manner as described above or in Example 1 below.

In certain embodiments, the present disclosure provides a pharmaceutical composition comprising barley extract as an active ingredient, optionally admixed with a pharmaceutically acceptable carrier or excipient or diluent for (i) preventing or treating a cognitive deficit, (ii) preventing or treating a condition, a disease, or a disorder, wherein inhibition of JNK provides a benefit, and/or iii) preventing or treating a condition, a disease, or a disorder associated with Tau-protein activity, in a subject.

The present disclosure also provides methods for formulating the disclosed compositions for pharmaceutical administration.

The compositions and methods of the present disclosure may be utilized to treat a subject in need thereof. In certain embodiments, the subject is a mammal such as a human or a non-human mammal. When administered to an animal, such as a human, the composition or the barley extract is preferably administered as a pharmaceutical composition comprising, for example, barley extract of the present disclosure and a pharmaceutically acceptable carrier. Pharmaceutically acceptable carriers are well known in the art and include, for example, aqueous solutions such as water or physiologically buffered saline or other solvents or vehicles such as glycols, glycerol, and oils such as olive oil or injectable organic esters. In certain embodiments, when such pharmaceutical compositions are for human administration, particularly for invasive routes of administration (i.e., routes, such as injection or implantation, that circumvent transport or diffusion through an epithelial barrier), the aqueous solution is pyrogen-free or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, and lyophile for reconstitution, powder, solution, syrup, suppository, injection, or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin or as an eye drop). The barley extract may also be formulated for inhalation. In yet another embodiment, barley extract may be simply dissolved or suspended in sterile water or double distilled water (DDW).

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of the active ingredient that can be combined with a carrier material to produce a single dosage form will generally be that amount of the compound which produces a therapeutic effect. Generally, out of one hundred percent, this amount will range from about 1% to about 99% of active ingredient, from about 5% to about 70%, or from about 10% to about 30%.

Methods of preparing these formulations or compositions include the step of bringing into association an active ingredient, such as barley extract of the present disclosure, with the carrier and, optionally, one or more accessory ingredients. In general, the formulations are prepared by uniformly and intimately bringing into association barley extract of the present disclosure with liquid carriers or finely divided solid carriers or both and then, if necessary, shaping the product.

Formulations of the present disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules or as a solution or a suspension in an aqueous or non-aqueous liquid or as an oil-in-water or water-in-oil liquid emulsion or as an elixir or syrup or as pastilles (using an inert base, such as gelatin and glycerin or sucrose and acacia) and/or as mouth washes and the like, each containing a predetermined amount of barley extract of the present disclosure as an active ingredient.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate and/or any of the following: (1) fillers or extenders, such as starches, lactose, sucrose, glucose, mannitol and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter or by incorporating sterilizing agents in the form of sterile solid compositions that can be dissolved in sterile water or some other sterile injectable medium immediately before use. These compositions may also optionally contain opacifying agents and may be of a composition that they release the active ingredient(s) only or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, suspensions, solutions, syrups, and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active ingredient, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth and mixtures thereof.

Formulations of the pharmaceutical compositions for rectal, vaginal or urethral administration may be presented as a suppository, which may be prepared by mixing one or more active compounds with one or more suitable nonirritating excipients or carriers comprising, for example, cocoa butter, polyethylene glycol, a suppository wax or a salicylate and which is solid at room temperature, but liquid at body temperature and, therefore, will melt in the rectum or vaginal cavity and release the active compound.

Formulations of the pharmaceutical compositions for administration to the mouth may be presented as a mouthwash or an oral spray or an oral ointment.

Alternatively, or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams, or spray formulations containing such carriers as are known in the art to be appropriate.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier and with any preservatives, buffers or propellants that may be required.

The ointments, pastes, creams, and gels may contain, in addition to an active ingredient, excipients, such as animal and vegetable fats, oils, waxes, paraffins, starch, tragacanth, cellulose derivatives, polyethylene glycols, silicones, bentonites, silicic acid, talc and zinc oxide or mixtures thereof.

Powders and sprays can contain, in addition to an active ingredient, excipients such as lactose, talc, silicic acid, aluminum hydroxide, calcium silicates and polyamide powder or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of barley extract of the present disclosure to the body. Such dosage forms can be made by dissolving or dispersing the active ingredient in the proper medium. Absorption enhancers can also be used to increase the flux of the active ingredient across the skin. The rate of such flux can be controlled by either providing a rate controlling membrane or dispersing the compound in a polymer matrix or gel.

Ophthalmic formulations, eye ointments, powders, solutions, and the like, are also contemplated as being within the scope of this invention. If desired, liquid ophthalmic formulations have properties similar to that of lacrimal fluids, aqueous humor or vitreous humor or are compatable with such fluids. A preferred route of administration is local administration (e.g., topical administration, such as eye drops or administration via an implant).

The phrases “parenteral administration” and “administered parenterally” as used herein means modes of administration other than enteral and topical administration, usually by injection and includes, without limitation, intracerebroventricular (ICV), intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise barley extract as described herein in combination with one or more pharmaceutically acceptable sterile isotonic aqueous or nonaqueous solutions, dispersions, suspensions or emulsions or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol and the like) and suitable mixtures thereof, vegetable oils, such as olive oil and injectable organic esters, such as ethyl oleate. Proper fluidity can be maintained, for example, by the use of coating materials, such as lecithin, by the maintenance of the required particle size in the case of dispersions and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid and the like. It may also be desirable to include isotonic agents, such as sugars, sodium chloride and the like into the compositions. In addition, prolonged absorption of the injectable pharmaceutical form may be brought about by the inclusion of agents that delay absorption such as aluminium monostearate and gelatin.

In some cases, in order to prolong the effect of active ingredient, it is desirable to slow the absorption of the active ingredient from subcutaneous or intramuscular injection. This may be accomplished by the use of a liquid suspension of crystalline or amorphous material having poor water solubility. The rate of absorption of the active ingredient then depends upon its rate of dissolution, which, in turn, may depend upon crystal size and crystalline form. Alternatively, delayed absorption of a parenterally administered active ingredient form is accomplished by dissolving or suspending the drug in an oil vehicle.

For use in the methods of this disclosure, active ingredient can be given per se or as a pharmaceutical composition containing, for example, 0.1 to 99.5% of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the active ingredient, the route of administration, the time of administration, the rate of excretion of the active ingredient being employed, the duration of the treatment, other drugs, compounds and/or materials used in combination with the particular compound(s) employed, the age, sex, weight, condition, general health and prior medical history of the patient being treated and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of an active ingredient that is sufficient to elicit the desired therapeutic effect. It is generally understood that the effective amount of the active ingredient will vary according to the weight, sex, age, and medical history of the subject. Other factors which influence the effective amount may include, but are not limited to, the severity of the patient's condition, the disorder being treated, the stability of the active ingredient and, if desired, another type of therapeutic agent being administered with the active ingredient (barley extract) of the present disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active ingredient used in the compositions and methods of the present disclosure will be that amount of the active ingredient that is the lowest dose effective to produce a therapeutic effect. Such an effective dose will generally depend upon the factors described above.

If desired, the effective daily dose of the active ingredient may be administered as one, two, three, four, five, six or more sub-doses administered separately at appropriate intervals throughout the day, optionally, in unit dosage forms. In certain embodiments, the active ingredient may be administered two or three times daily. In some instances, the active ingredient may be administered once daily.

The patient receiving this treatment is any animal in need, including primates, in particular humans and other mammals such as equines, cattle, swine and sheep; and poultry and pets in general.

Wetting agents, emulsifiers, and lubricants, such as sodium lauryl sulfate and magnesium stearate, as well as coloring agents, coating agents, release agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol and the like; and (3) metal-chelating agents, such as citric acid, ethylenediamine tetraacetic acid (EDTA), sorbitol, tartaric acid, phosphoric acid and the like.

The present disclosure further provides a pharmaceutical composition comprising barley extract as an active ingredient for use in the manufacture of a medicament.

In certain embodiments, the present disclosure provides use of a pharmaceutical composition comprising barley extract as an active ingredient in the manufacture of a medicament for preventing or treating a cognitive deficit in a subject. In some embodiments, the cognitive deficit is protein phosphatase inhibitor induced cognitive deficit. In some instances, the protein phosphatase inhibitor is okadaic acid (OKA) or a pharmaceutically acceptable salt thereof. In certain embodiments, the cognitive deficit is Alzheimer's disease (AD) or Parkinson's disease. In some embodiments, the cognitive deficit is Alzheimer's disease (AD). In certain embodiments, the barley extract is obtained by the method described in Indian U.S. patent number 236375, the entirety of which is incorporated herein by reference.

In certain embodiments, the present disclosure provides use of a pharmaceutical composition comprising barley extract as an active ingredient in the manufacture of a medicament for preventing or treating a condition, a disease, or a disorder, wherein inhibition of JNK provides a benefit. In certain embodiments, the condition, the disease, or the disorder is cognitive deficit. In some embodiments, the cognitive deficit is Alzheimer's disease (AD) or Parkinson's disease. In some instances, the cognitive deficit is Alzheimer's disease (AD). In certain embodiments, the barley extract is obtained by the method described in Indian U.S. patent number 236375, the entirety of which is incorporated herein by reference.

In certain embodiments, the present disclosure provides use of a pharmaceutical composition comprising barley extract as an active ingredient in the manufacture of a medicament for preventing or treating a condition, a disease, or a disorder associated with the activity of Tau-protein, in a subject. In certain embodiments, the condition, the disease, or the disorder is cognitive deficit. In some embodiments, the cognitive deficit is Alzheimer's disease (AD) or Parkinson's disease. In some instances, the cognitive deficit is Alzheimer's disease (AD). In certain embodiments, the barley extract is obtained by the method described in Indian U.S. patent number 236375, the entirety of which is incorporated herein by reference.

The present disclosure also provides a method for preventing or treating a cognitive deficit in a subject. The method comprises administering a therapeutically effective amount of a barley extract as an active ingredient or a pharmaceutical composition comprising barley extract as an active ingredient to the subject in need thereof. In certain embodiments, the cognitive deficit is associated with the activity of Tau-protein, and/or JNK. In certain embodiments, the cognitive deficit is protein phosphatase inhibitor induced cognitive deficit. In some instances, the protein phosphatase inhibitor is okadaic acid (OKA) or a pharmaceutically acceptable salt thereof. In some instances, the cognitive deficit is Alzheimer's disease (AD) or Parkinson's disease. In certain embodiments, the barley extract is obtained by the method described in Indian patent number 236375, the entirety of which is incorporated herein by reference.

In certain embodiments, the present disclosure provides a method for preventing or treating a condition, a disease, or a disorder in a subject, wherein inhibition of JNK provides a benefit. The method comprises administering a therapeutically effective amount of a barley extract as an active ingredient or a pharmaceutical composition comprising barley extract as an active ingredient to the subject in need thereof. In certain embodiments, the condition, the disease, or the disorder is cognitive deficit. In some embodiments, the cognitive deficit is Alzheimer's disease (AD) or Parkinson's disease. In some instances, the cognitive deficit is Alzheimer's disease (AD). In certain embodiments, the barley extract is obtained by the method described in Indian patent number 236375, the entirety of which is incorporated herein by reference.

The present inventors further found that the barley extract as provided herein increases activation of BDNF (Brain-Derived Neurotrophic Factor) and decreases activation of NF-κβ (Nuclear factor kappa B). Thus, in certain embodiments, the pharmaceutical composition as provided herein may be useful in the treatment of conditions associated with BDNF activity and/or NF-κβ activity.

The pharmaceutical composition of the present disclosure may be used alone (monotherapy) or conjointly with one or more other methods/compositions (conjoint therapy).

The dosage of the active ingredient (barley extract) of the present disclosure varies depending on a patient's age, weight, or symptoms, as well as the compound's potency or therapeutic efficacy, the dosing regimen and/or treatment time. Generally, suitable routes of administration may, for example, include oral, eyedrop, rectal, transmucosal, topical or intestinal administration; parenteral delivery, including intramuscular, subcutaneous, intramedullary injections, as well as intrathecal, direct intraventricular, intravenous, intraperitoneal, intranasal, or intraocular injections. The barley extract of the present disclosure may be administered in an amount of 0.2 mg, 0.5 mg or 1 mg up to 500 mg, 1 g, or 2 g per dosage regimen. The dosage may be administered once per week, once per three days, once per two days, once per day, twice per day, three times per day or more often. In alternative embodiments, in certain adults the barley extract can be continuously administered by intravenous administration for a period of time designated by a physician. Since the dosage is affected by various conditions, an amount less than or greater than the dosage ranges contemplated about may be implemented in certain cases. A physician can readily determine the appropriate dosage for a patient undergoing therapeutic treatment.

In certain embodiments, the pharmaceutical composition of the present disclosure may further comprise an additional active ingredient having an effect of treating a condition, a disease or a disorder as described above. In certain embodiments, the additional active ingredient may be selected from a group comprising a JNK inhibitor, a BDNF activator, and an NF-κB inhibitor. The dosage of the additional active ingredient can be a dosage that has been clinically used or may be a reduced dosage that is effective when administered in combination with barley extract of the present disclosure. The ratio of the barley extract of the present disclosure and the other additional active ingredient can vary according to age and weight of a subject to be administered, administration method, administration time, disorder to be treated, symptom and combination thereof. For example, the other additional active ingredient may be used in an amount of 0.01 to 100 parts by mass, based on 1 part by mass of the barley extract of the present disclosure.

The present disclosure now being generally described, it will be more readily understood by reference to the following examples, which are included merely for purposes of illustration of certain aspects and embodiments of the present disclosure and are not intended to limit the present disclosure in any way.

### EXAMPLES

### Example 1: Preparation of Barley Extract

Barley flour 1 kg obtained by grinding barley grains is taken in a glass vessel fitted with standard joint and added 2 litres of double distilled water with proper stirring followed by vigorous shaking. The glass vessel is then property covered and kept in an incubator at 27±3° C. for 16 hours. After mat the cover of glass vessel was removed, and the vessel was put on a heating system (rotamentle fitted with heating element and thermostat having insulation of glass wool so as to avoid direct contact of heat with glass flasks) vessel is fitted with distillation set having proper cooling system. Optimum temperature at the time of distillation was 110+30° C. The liquid so obtained during distillation process was passed over cotton bed (fitted in funnels) and collected in the flasks (3-4 fractions, each of approx 400 ml). These fractions were property covered and kept at a temperature of 10±3° C. for one hour. The flasks were removed to the normal room temperature and varies from time to time with seasons. The contents of the flasks were mixed and filtered over a bed of cotton. The filtrate (solution or the extract) thus obtained is the pharmaceutical preparation, desired to be prepared. This solution or the extract was named as “SAAC” (also referred to interchangeably herein as “SACC”). The prepared extract is stored at 4-8° C. for a longer shelf-life.

### Material and Methods

### Animals

Adult male Wistar rats (200-230 g, 2 months old) were used.

### Drugs & treatment

Nuclear factor kappa-light-chain-enhancer of activated B cells (NF-KB) and BDNF ELISA kits were procured from Imegenex, San Diego, USA and Millipore, USA respectively. All other chemicals used for biochemical estimations were of analytical grade. SAAC was dissolved in double distilled water (DDW) while okadaic acid was dissolved in artificial cerebrospinal fluid (aCSF) (2.9 mM KCl, 147 mM NaCl, 1.7 mM CaCl₂, 1.6 mM MgCl₂, and 2.2 mM D-glucose).

### Drug Dilution

$$SAAC ( 1.5 ml ) + D D W 3.5 ml$$

### Drug Solution was Freshly Prepared and Administered in a Constant Volume of 5 ml/kg Body Weight

### Surgical Procedures: ICV Injection of OKA

Intracerebroventricular injection of okadaic acid was performed according to the procedure of Kamat et al. (2010). Rats were anesthetized with thiopentone (Neon Laboratories, India, 45 mg/kg, i.p.). The scalp was shaved, cleaned, and cut to expose the skull. The head was positioned in a stereotaxic frame and a midline sagittal incision was made in the scalp. Burr holes were drilled in the skull on both sides over the lateral ventricles by using the following coordinates: 0.8 mm posterior to bregma; 1.5 mm lateral to sagittal suture and 3.6 mm beneath the surface of the brain (Paxinos et al., 1980). Okadaic acid (200 ng) was injected bilaterally making the total dose of 100 ng in each ventricle. The concentration of okadaic acid in aCSF was adjusted so as to deliver 4 μl of the solution. Sham animals received ICV injection of the same volume of aCSF. The skin was sutured after injection followed by daily application of antiseptic powder (Neosporin). Postoperatively, the rats were fed with oral glucose and normal pellet diet for 4 days, followed by normal pellet diet alone.

### Experimental Design

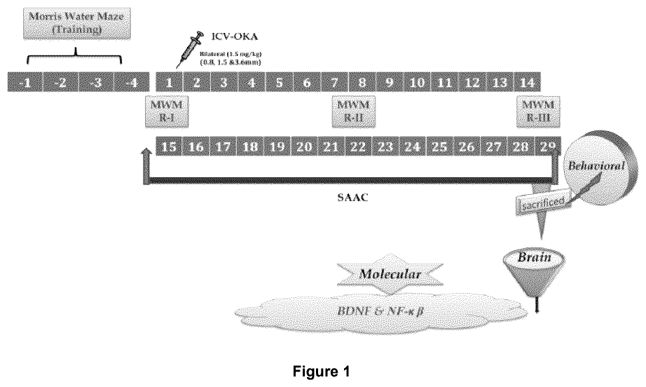

The experimental design is illustrated in FIG. 1. Rats were randomly assigned to four different groups containing 7 animals in each group viz Group 1: sham-operated animals received aCSF (4 μl, each side) through ICV route; Groups 2: animals received ICV-OKA (100 ng each ventricle) prepared in aCSF; Groups 3: ICV-OKA (100 ng each ventricle) rats being administered SAAC (i.p.) respectively for 28 days; Groups 4: ICV-OKA rats being administered SAAC (oral) for 28 days. Memory impairment was assessed by Morris Water Maze on days 15^{th}, 21^{st}, 28^{th}. After behavioral experiments, rats were anesthetized with thiopentone sodium (40 mg/kg; i.p.) and blood was collected through tail vein followed by decapitation of animals by cervical dislocation. Brains were quickly removed, cleaned with chilled saline, and stored at −80° C.

### Assessment of Cognitive Performance in Morris Water Maze Task

The acquisition and retention of memory was evaluated by using Morris water maze (Morris et al., 1982; Tuzcu and Baydas, 2006). The apparatus consisted of a circular water tank (180 cm in diameter and 60 cm high). A platform (12.5 cm in diameter and 38 cm high) invisible to the rats, was set 2 cm below the water level inside the tank with water maintained at 28.5±2° C. at a height of 40 cm. The tank was located in a large room where there were several brightly colored cues external to the maze; these were visible from the pool and could be used by the rats for spatial orientation. The position of the cues remained unchanged throughout the study. The water maze task was carried out for four consecutive days. The rats received four consecutive daily training trials in the following 4 days, with each trial having a ceiling time of 90 s and a trial interval of approximately 30 s. For each trial, each rat was put into the water at one of four starting positions, the sequence of which being selected randomly. During test trials, rats were placed into the tank at the same starting point, with their heads facing the wall. The rat had to swim until it climbed onto the platform submerged underneath the water. After climbing onto the platform, the animal remained there for 20 s before the commencement of the next trial. The escape platform was kept in the same position relative to the distal cues. If the rat failed to reach the escape platform within the maximally allowed time of 90 s, it was guided with the help of a rod and allowed to remain on the platform for 20 s. During acquisition phase day (day 15, 22 and 29) the time to reach the platform (escape latency in seconds) was measured by using computer tracking system with EthoVision software (Noldus Information Technology, Wageningen, Netherlands) and (IAL; initial acquisition latency) was measured.

### Quantification of NF-KB p65 Unit

The NF-KB/p65 ActivELISA kit (Imgenex, USA) was used to measure NF-KB-free p65 in the nuclear fraction of different brain regions. The nuclear levels of p65 may correlate positively with the activation of the NF-KB pathway. The NF-KB ActivELISA is a sandwich ELISA in which free p65 was captured by anti-p65 antibody-coated plates and the amount of bound p65 was detected by adding a second anti-p65 antibody followed by alkaline phosphatase conjugated secondary antibody using colorimetric detection in an ELISA plate reader at 405 nm. The results were expressed as ng/mg of protein.

### Quantification of Brain derived Neurotrophic Factors (BDNF)

The quantification of BDNF was done with the help and instructions provided by Millipore, USA quantikine rat BDNF immunoassay kit.

### Results

### Behavioral Observations Effect of SAAC on Cognitive Function

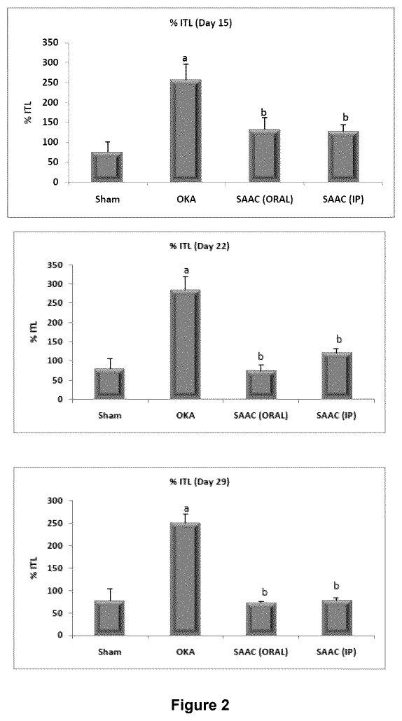

The cognitive function was assessed in the Morris water maze test. Two-way ANOVA followed by Tukey's test was employed to discover the inter-group variation in escape latency of Morris water maze by considering day of testing and treatment as two independent variables. The mean escape latency did not differ between any of the groups on the first day of training session in Morris water maze but from second day onwards, there was significant difference in escape latency. ICV-OKA rats showed 1.5-fold lower ability to find the platform and learn its location on 15^{th} day in morris water maze task. This poor performance was significantly (p<0.05) mitigated by the chronic treatment with SAAC. However, SAAC treatment by oral and i.p. route did not show any significant difference. The treatment effect of SAAC on % Initial Transfer Latency (% ITL) of ICV-OKA rats in Morris water maze is illustrated in FIG. 2. Values are expressed as mean±SEM; and a represents difference from sham group (p<0.05); and b represents difference from ICV-OKA administered group (p<0.05).

### Effect of SAAC on Hippocampal NFκβ Activity

NF-κβ p65 subunit was significantly (105%) elevated in hippocampus of ICV-OKA administered rats. Both oral and i.p. administration of SAAC significantly inhibited enhanced hippocampal NF-κβ p65 subunit expression 16% and 24% respectively as compared to ICV-OKA administered rats.

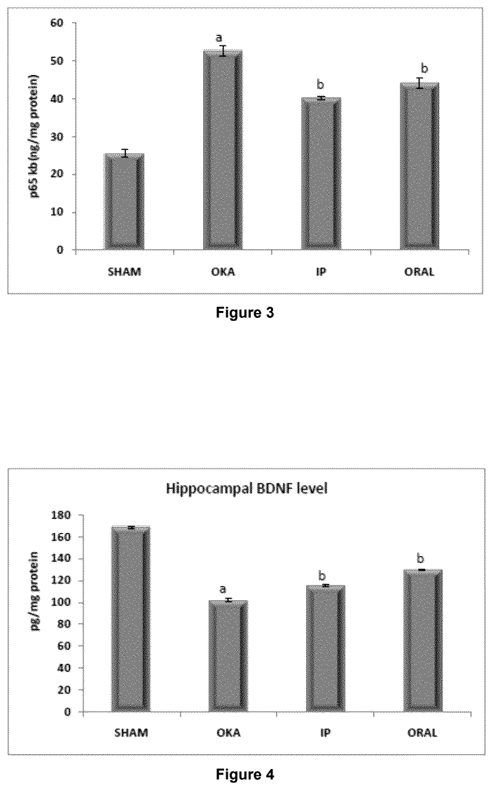

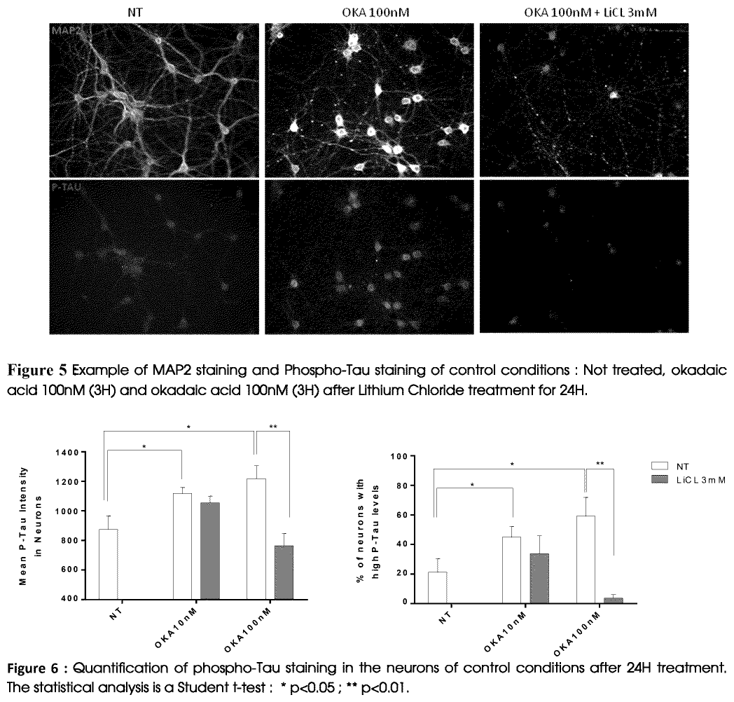

The treatment effect of SAAC on NF-Kβ activity in ICV-OKA rats is illustrated in FIG. 3. Values are expressed as mean±SEM; a represents difference from sham group (p<0.05); b represents difference from ICV-OKA administered group (p<0.05).

### Effect of SAAC on Hippocampal BDNF Level

A 40% decrease in brain derived neurotrophic factors was observed in the ICV-OKA rats as compared to sham group rats. SAAC treatment with both oral and i.p. route increased the BDNF level in the hippocampus 27% and 13% respectively as compared to ICV-OKA rats. However, no significance was observed between oral and i.p. group animals. The treatment effect of SAAC on BDNF level in ICV-OKA rats is illustrated in FIG. 4. Values are expressed as mean±SEM; a represents difference from sham group (p<0.05); and b represents difference from ICV-OKA administered group (p<0.05).

### Example 2: Evaluation of the Neuroprotective Potential of a Test Compound in Relation With Alzheimer's Disease

The potential efficacy of the test compound (SAAC) against Alzheimer's disease was evaluated using an in vitro model in which Tau hyper-phosphorylation Is Induced by pharmacological treatment.

### 2.1 Test Compounds

The test compound was tested in culture medium at 0.01, 0.03, 0.1, 0.3, 1.3, and 10%. Positive control of reversion of hyperphosphorylation was UCI at 3 mM.

### 2.2. Biological System

Primary cortical cultures from E 19 rat embryos.

### 2.3. Assay Endpoints

Phospho-Tau intensity in neurons and percentage of neurons with hyperphosphorylated Tau.

### 2.4. Experimental Procedure

Cortical neurons from E 19 rat embryos were harvested and plated in 96 well plates. After 9 days in vitro, cells were treated with LiCI at 3 mM for 24 hours as a positive neuroprotective control or the test compound (SAAC) at 7 different concentrations. Another plate was pre-treated with the test compound (SAAC) at 7 different concentrations for 4 hours.

At the end of treatment durations, cells were treated with okadaic acid (OKA) for 3 hours. The cells were then fixed and processed for immunofluorescence analysis of Phospho-Tau (P-Tau) in neurons (Phospho-Tau labelling and neuronal labelling with MAP2 antibody).

### Results

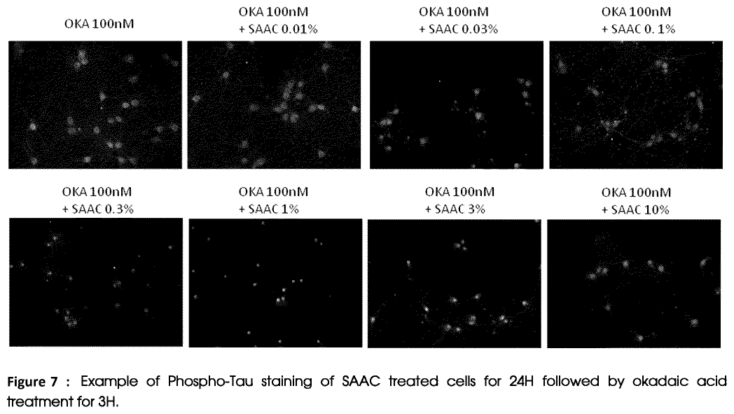

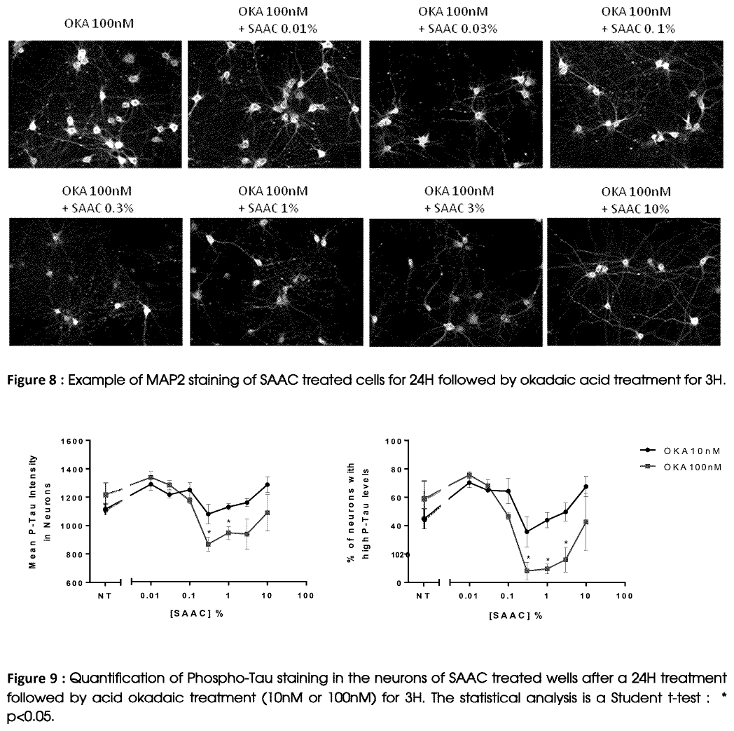

After 9 days in vitro, neurons were treated with LiCI at 3 mM for 24 hours as a positive neuroprotective control (FIG. 5) or the test compound (SAAC) at 7 different concentrations (FIG. 7 & FIG. 8). At the end of treatment duration, cells were treated with okadaic acid (OKA) for 3 hours. After Phospho-Tau and MAP2 labelling, the signals were acquired and the mean intensity of Phospho-Tau staining in the neurons as well as the percentage of neurons with a high Phospho-Tau level was assessed.

The okadaic acid treatment at 10 nM and 100 nM increased the phosphorylation of the Tau protein in a dose dependent way. There is almost a 2-fold (for 10 nM okadaic acid) and 3-fold (for the 100 nM okadaic acid) increase in the percentage of neurons with high Phospho-Tau levels.

The lithium slightly reduced the phosphorylation of Tau after a 10 nM okadaic acid treatment, while for the 100 nM okadaic acid treatment, the lithium entirely reversed the phosphorylation of Tau coming back to levels lower than the non-treated neurons.

Reversion of the Tau phosphorylation with 24 h treatment of SAAC was then analysed. The SAAC dose response on Tau-Phosphorylalion and MAP2 associated staining are shown in FIG. 7 and FIG. 8, respectively.

The analysis of the Tau phosphorylation on neurons was performed. The results are shown in FIG. 9.

The phosphorylation of Tau was prevented at 0.3%, 1% and 3% after a 100 nM okadaic acid treatment. The same effect was not observed with 10 nM okadaic acid treatment (at least not statistically significant).

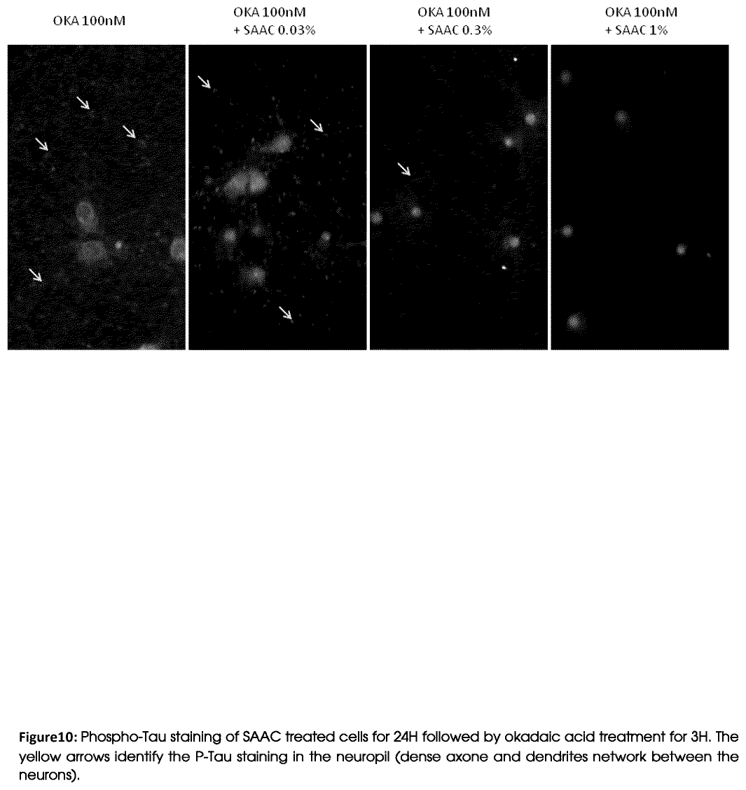

These analyses were mode on the soma of the neurons but it is noticed that SAAC (as well as the lithium) was able to prevent the phosphorylation of Tau in the neuropil (FIG. 10). The okadaic acid treatment greatly increased the amount of phosphorylated tau in the axons and dendrites of the neurons (yellow arrows in FIG. 10). This signal decreased in a dose dependent way when the neurons were treated with SAAC reinforcing the protective status of SAAC treatment assessed in the neurons' soma.

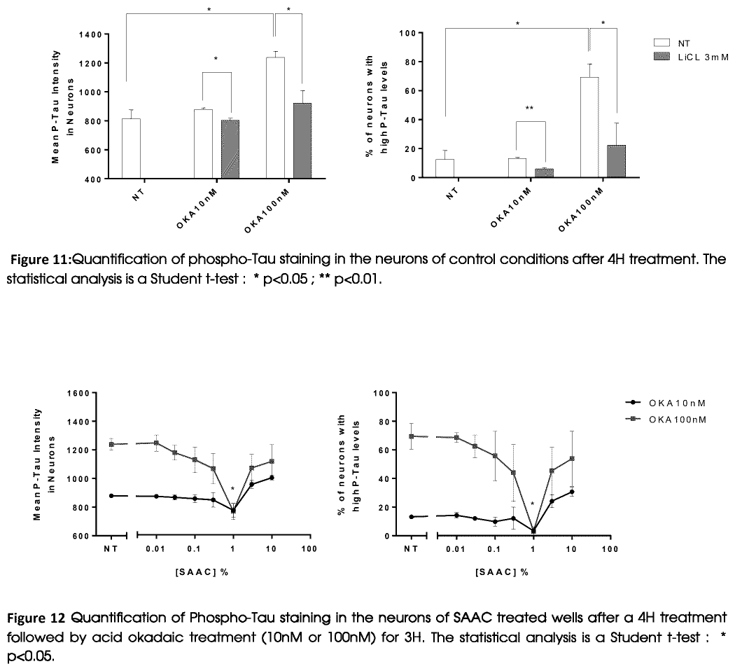

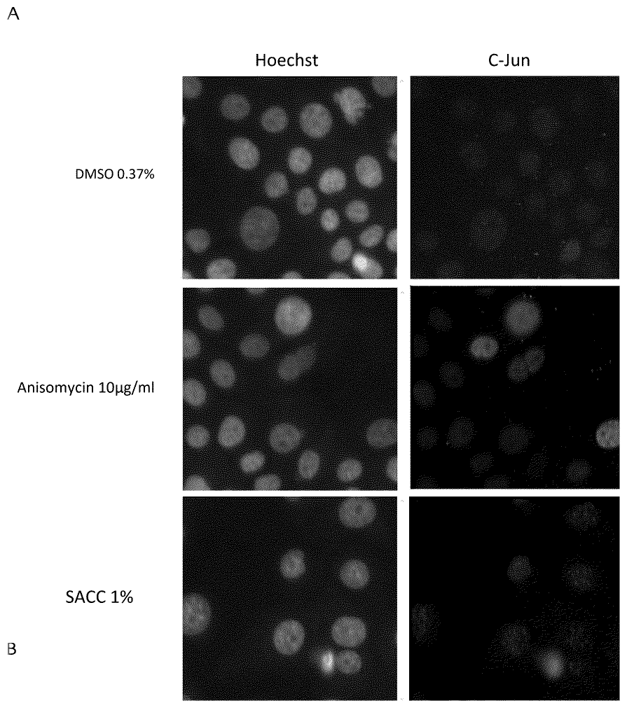

In parallel, also performed the same experiment with a 4 h treatment of lithium and SAAC using still 10 nM and 100 nM of okadaic acid to increase the phosphorylation of Tau. The results of control treatment and SAAC treatment are shown in FIG. 11 and FIG. 12, respectively.

In this experiment, the 10 nM concentration of okadaic acid was not able to elicit any increase in the phosphorylation of Tau. However, a dose response protection effect of SAAC treatment on the 100 nM concentration of okadaic acid is observed. The 1% SAAC concentration was the only one where the protection against Tau Phosphorylation was statistically significant.

The data demonstrates that the okadoic acid induced a high phosphorylation of Tau at 100 nM, and a mild to slight increase to 10 nM. SAAC did have a protective effect against Tau phosphorylation in neurons at 0.3%, 1%, and 3% after a 24 H pre-treatment. The 1% concentration was also effective after a 4 h treatment. The SAAC also induced a great decrease in tau phosphorylation in the axons and dendrites of the neurons reinforcing its protective status found in the neuron soma.

### Example 3: Evaluation of SACC Effect on Phosphorylation Level of the c-Jun (Substrate of JNK)

SACC was received as an aqueous stock solution. The different solutions were created by performing cascade dilutions in the culture medium.

TABLE 1
Dose range tested for SACC

| Tested compound | Tested dilutions (v/v in the culture medium) |
|---|---|
| SACC | 0; 0.1; 0.3; 1% |

Cells were seeded at 12 10³ cells/well in 96 well plates and cultured for 24 h. Subsequently, cells were exposed to the different tested conditions (reference treatment: 1 μM anisomycin 10 μg/mL, 30 min). At the end of the treatment, the cells were processed for immunofluorescence with a specific antibody against the phosphorylated form of c-Jun. The fluorescent immune-staining was subsequently imaged by high content imaging. Single cell analysis of the phospho c-Jun immune-staining was performed.

Based on the bell-shaped dose-response function related to the cytotoxic effect of the SACC, a 1% concentration of SACC was used to evaluate the JNK signaling pathway's response to the effect of SACC using molecular HCA assays.

The pharmacological modulators that decreased the cytotoxic effects of 1% SACC did it on the 1% were maximally effective at 50 h treatment. Therefore, the effect of SACC was studied after 50 h treatment with 0.1%; 0.3% and 1% SACC on phosphorylation level of the c-Jun (substrate of JNK).

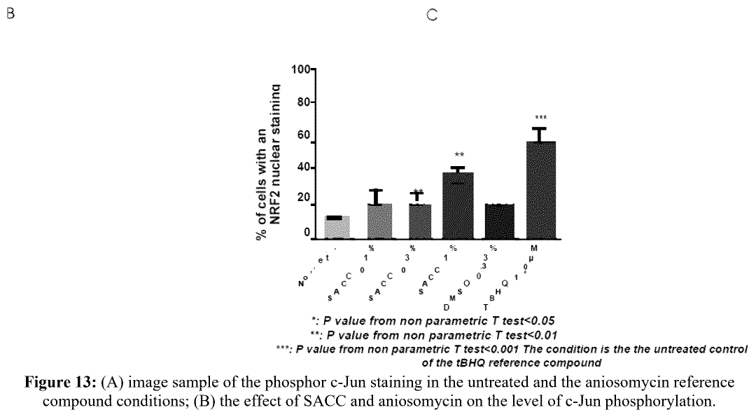

FIG. 13(A) shows image sample of the phosphor c-Jun staining in the untreated and the aniosomycin reference compound conditions; while 13(B) shows the effect of SACC and aniosomycin on the level of c-Jun phosphorylation.

Aniosomycin induced c-Jun phosphorylation in the total population of treated cells. At the highest tested dose, the SACC induced a weak but a significant c-Jun phosphorylation in a subset of the cell population. Noted the intranuclear dotted distribution of phosphor c-Jun in SACC treated cells.

1. A composition comprising barley extract as an active ingredient for use in preventing or treating a cognitive deficit in a subject.

2. The composition as claimed in claim 1, wherein the barley extract is obtained by the process comprising the steps of mixing barley grain flour with distilled water to get a mixture; stirring the mixture at an ambient temperature; and distilling the mixture to obtain a distilled extract; and filtering the distilled extract to obtain the barley extract.

3. The composition as claimed in claim 2, wherein the mixture is kept at a temperature of about 27±3° C. for about 16±2 hours.

4. The composition as claimed in claim 2, wherein the mixture is distilled at a temperature of about 110±30° C. to obtain a distilled extract.

5. The composition as claimed in claim 4, wherein the distilled extract is kept at about 10±3° C. for about 1 h.

6. The composition as claimed in claim 1, wherein the composition is a pharmaceutical composition or a food composition.

7. The composition as claimed in claim 1, wherein the cognitive deficit is protein phosphatase inhibitor induced cognitive deficit.

8. The composition as claimed in claim 7, wherein the protein phosphatase inhibitor is okadaic acid (OKA) or a pharmaceutically acceptable salt thereof.

9. The composition as claimed in claim 1, wherein the cognitive deficit is Alzheimer's disease (AD) or Parkinson's Disease.

10. The composition as claimed in claim 1, wherein the cognitive deficit is Alzheimer's disease (AD).

11. The composition as claimed in claim 1, wherein the cognitive deficit is associated with the activity of Tau-protein, and/or JNK.

12. The composition as claimed in claim 1, wherein the barley extract increases the activity of BDNF (Brain Derived Neurotrophic Factor) and/or decreases the activity of NF-κB (Nuclear factor kappa B).

13. A method for preventing or treating a cognitive deficit in a subject; wherein the method comprises administering an effective amount of a barley extract as an active ingredient or a composition comprising barley extract as an active ingredient to the subject in need thereof.

14. The method as claimed in claim 13, wherein the barley extract is obtained by the process comprising the steps of mixing barley grain flour with distilled water to get a mixture; stirring the mixture at an ambient temperature; and distilling the mixture to obtain the barley extract.

15. The method as claimed in claim 14, wherein the mixture is kept at a temperature of about 27±3° C. for about 16±2 hours.

16. The method as claimed in claim 14, wherein the mixture is distilled at a temperature of about 110±30° C. to obtain the barley extract.

17. The method as claimed in claim 16, wherein the barley extract obtained is kept at about 10±3° C. for about 1 h.

18. The method as claimed in claim 13, wherein the composition is a pharmaceutical composition or a food composition.

19. The method as claimed in claim 13, wherein the cognitive deficit is protein phosphatase inhibitor induced cognitive deficit.

20. The method as claimed in claim 19, wherein the protein phosphatase inhibitor is okadaic acid (OKA) or a pharmaceutically acceptable salt thereof.

21. The method as claimed in claim 12, wherein the cognitive deficit is Alzheimer's disease (AD) or Parkinson's Disease.

22. The method as claimed in claim 13, wherein the cognitive deficit is Alzheimer's disease (AD).

23. The method as claimed in claim 13, wherein the cognitive deficit is associated with the activity of Tau-protein, and/or JNK.

24. The method as claimed in claim 13, wherein the barley extract increases the activity of BDNF and decreases the activity of NF-κβ.

25. The method as claimed in claim 13, wherein the composition is a pharmaceutical composition or a food composition.

26. Use of a composition comprising barley extract as an active ingredient for use in preventing or treating a cognitive deficit in a subject.

27. The use as claimed in claim 26, wherein the barley extract is obtained by the process comprising the steps of mixing barley grain flour with distilled water to get a mixture; stirring the mixture at an ambient temperature; and distilling the mixture to obtain the barley extract.

28. The use as claimed in claim 27, wherein the mixture is kept at a temperature of about 27±3° C. for about 16±2 hours.

29. The use as claimed in claim 27, wherein the mixture is distilled at a temperature of about 110±30° C. to obtain the barley extract.

30. The use as claimed in claim 29, wherein the barley extract obtained is kept at about 10±3° C. for about 1 h.

31. The use as claimed in claim 26, wherein the composition is a pharmaceutical composition or a food composition.

32. The use as claimed in claim 26, wherein the cognitive deficit is protein phosphatase inhibitor induced cognitive deficit.

33. The use as claimed in claim 26, wherein the protein phosphatase inhibitor is okadaic acid (OKA) or a pharmaceutically acceptable salt thereof.

34. The use as claimed in claim 26, wherein the cognitive deficit is Alzheimer's disease (AD) or Parkinson's Disease.

35. The use as claimed in claim 26, wherein the cognitive deficit is Alzheimer's disease (AD).

36. The use as claimed in claim 26, wherein the cognitive deficit is associated with the activity of Tau-protein, and/or JNK.

37. The use as claimed in claim 26, wherein the barley extract increases the activity of BDNF and/or decreases the activity of NF-κβ.

38. The use as claimed in claim 26, wherein the composition is for the manufacture of a medicament for preventing or treating a cognitive deficit.
